(19) United States
(12) Patent Application Publication
(10) Pub. No.: US 2019/0151517 A1
(43) Pub. Date: May 23, 2019

(54) AURAL SUCTION DEVICE

(71) Applicant: MESHIL A.M.O.H. AL-JARBA, SAFAT (KW)

(72) Inventor: MESHIL A.M.O.H. AL-JARBA, SAFAT (KW)

(21) Appl. No.: 16/177,867

(22) Filed: Nov. 1, 2018

Related U.S. Application Data
(60) Provisional application No. 62/588,202, filed on Nov. 17, 2017.

(51) Int. Cl. A61M 1/00

(52) U.S. Cl. CPC A61M 1/0039; A61M 2210/0662; A61M 1/0043

(57) ABSTRACT

The aural suction device includes a rigid tubular handle, a flexible tube detachably connected to the tubular handle, and a safety stop adjustably secured to an outer surface of the flexible tube. The flexible tube can be inserted into the ear of a patient and the aural suction device can be connected to a suction source to remove liquid from the ear. The safety stop can be positioned on the flexible tube to limit a length of the flexible tube to be inserted into the ear. The length of the flexible tube inserted into the ear can be sufficient to provide appropriate suction without impacting the eardrum if the patient moves unexpectedly during the procedure.

### CROSS-REFERENCE TO RELATED APPLICATION

This application claims the benefit of U.S. Provisional Patent Application Ser. No. 62/588,202, filed Nov. 17, 2017.

### BACKGROUND

### I. Field

The disclosure of the present patent application relates to medical devices, and particularly, to an aural suction device with safety stop.

### 2. Description of the Related Art

It is a known medical practice to employ suction tubes for removing liquids from the ear. These suction tubes often include rigid portions that are inserted into the aural canals. When patients (particularly children) move during such procedures, the device can injure or puncture the eardrum or other parts of the ear, ear canal, or nasal cavity.

Thus a flexible aural suction tube with safety stop solving the aforementioned problems is desired.

### SUMMARY

The aural suction device includes a rigid tubular handle, a flexible tube detachably connected to the tubular handle, and a safety stop adjustably secured to an outer surface of the flexible tube. The flexible tube can be inserted into the ear of a patient and the aural suction device can be connected to a suction source to remove liquid from the ear. The safety stop can be positioned on the flexible tube to limit a length of the flexible tube to be inserted into the ear. The length of the flexible tube inserted into the ear should be sufficient to provide appropriate suction without impacting the eardrum if the patient moves unexpectedly during the procedure.

These and other features of the present disclosure will become readily apparent upon further review of the following specification and drawings.

### BRIEF DESCRIPTION OF THE DRAWINGS

FIG. 1 is an environmental, perspective view of an aural suction device.

FIG. 2 is a perspective view of an aural suction device.

FIG. 3 is a further perspective view of an aural suction device.

FIG. 4 is an exploded, perspective view of an aural suction device.

Similar reference characters denote corresponding features consistently throughout the attached drawings.

### DETAILED DESCRIPTION OF THE PREFERRED EMBODIMENTS

The aural suction device 5 includes a tubular handle 10, a flexible suction tube 20 detachably connected to the tubular handle 10, and a safety stop 22 detachably connected to an outer surface of the flexible suction tube 20. The tubular handle 10 can have a rigid peripheral wall and an opening 16 extending through a portion of the wall. The tubular handle 10 is configured for connection to a tube of a conventional suction machine. The opening 16 can facilitate manual control of the suction.

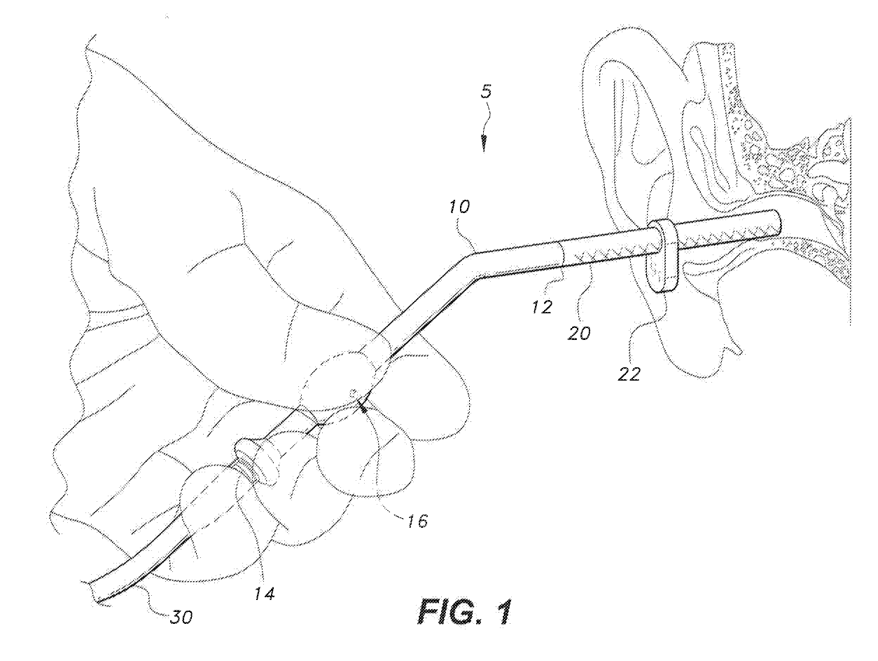

As shown in FIG. 1, the tubular handle 10 includes at least an open first end 12 and an open second end 14. The first end 12 can be detachably connected to flexible tube 20. The second end 14 is configured for attachment to a suction or vacuum source 30 to provide an aural suction system. The opening 16 is in fluid communication with the vacuum source 30 and the flexible tube 20, allowing for manual control of suction through the flexible tube 20. The rigid tubular handle 10 may be attached to the flexible tube 20 or the vacuum source 30 via one of a variety of means, including but not limited to a snug friction fit, a chemical adhesive, a threaded connection, or a coupling. The tubular handle 10 may be constructed from plastic, steel, or any other suitably rigid material.

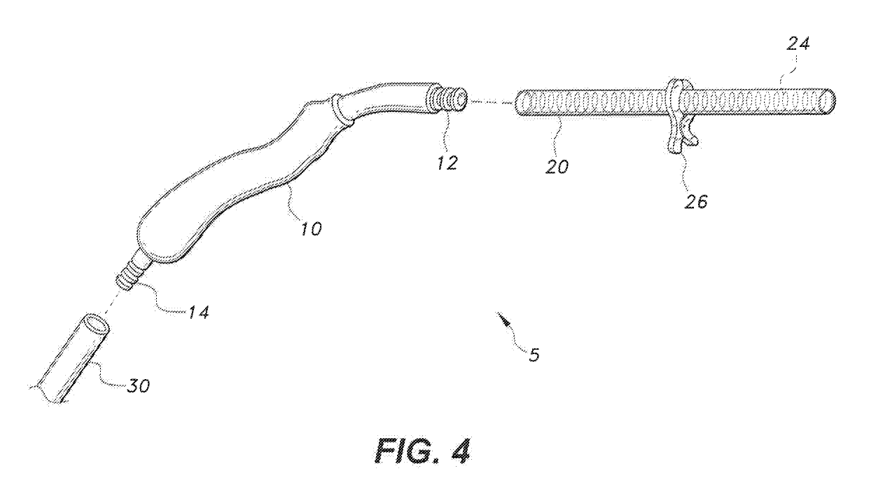

The flexible tube 20 can be made of any flexible material, such as rubber, plastic, or other suitable materials. As shown in FIG. 4, the flexible tube 20 can be reinforced with wire 24 to allow the flexible tube 20 to resist light pressures but bend in response to harder pressures, thereby reducing the risk of injury to the patient. The wire 24 can include a thin spiral wire embedded within the tube or a metal mesh.

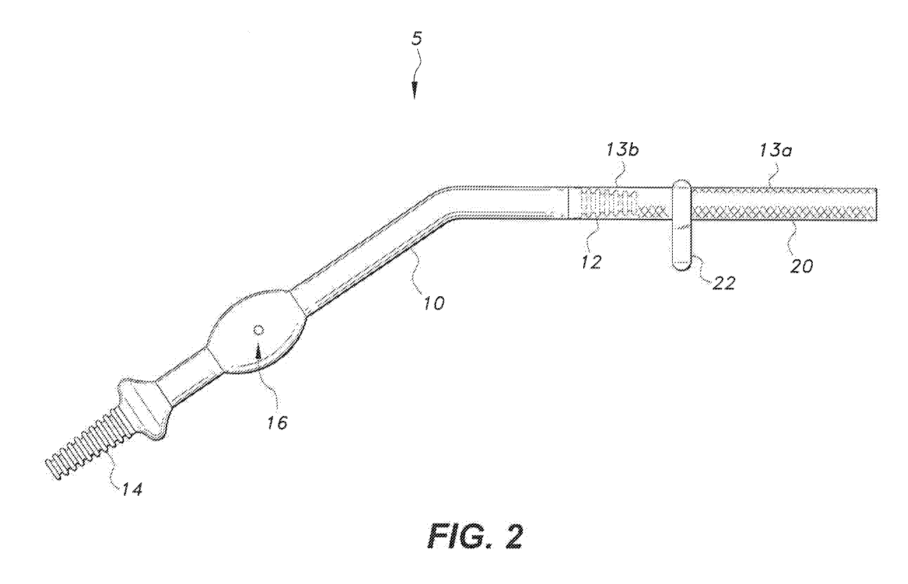

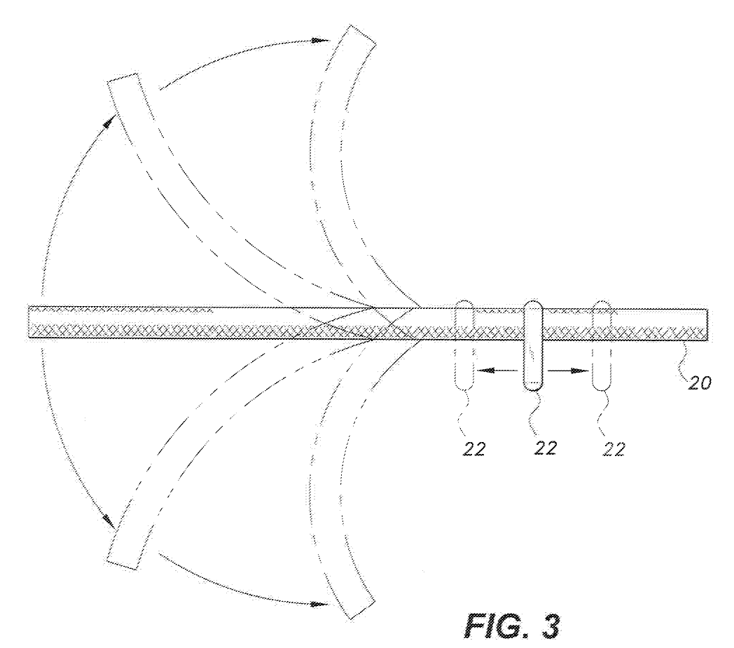

As shown in FIGS. 2 and 3, the safety stop 22 can be a ring or clamp configured to adjustably grip an outer peripheral portion of the flexible tube 20. Positioning the stop 22 on the tube 20 separates the flexible tube 20 into an ear insertable first portion 13a and a remaining second portion 13b. The safety stop 22 is configured to abut the outer ear when the ear insertable first portion 13a is inserted into the ear, and thereby prevent the remaining second portion 13b of the tube from entering the ear. A user can adjust the position of the safety stop 22 as needed to adjust the length of the ear insertable portion 13a (as shown in FIG. 3). The ear insertable portion can be long enough to provide appropriate suction without impacting the eardrum if the patient moves unexpectedly during the procedure.

As shown in FIG. 4, the releasable safety stop 22 may comprise a spring clamp 26. In an alternative embodiment shown in FIGS. 1-3, the releasable safety stop 22 may include a ring having a hole of approximately equal diameter to the outside diameter of the flexible tube 20, allowing the safety stop 22 to releasably engage the flexible tube 20.

The inner diameter of the flexible tube 20 can be essentially constant. The inner diameter of the rigid tubular handle 10 can be essentially constant or variable.

It is to be understood that the aural suction device is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. An aural suction device, comprising:
a tubular handle having first and second open ends and an opening between the first and second open ends;
a flexible tube detachably connected to the first end of the tubular handle; and
a safety stop adjustably secured to an outer surface of the flexible tube.

2. The aural suction device according to claim 1, further comprising a wire embedded within the flexible tube.

3. The aural suction device according to claim 2, wherein the wire embedded within the flexible tube comprises a spiral wire.

4. The aural suction device according to claim 2, wherein the wire embedded within the flexible tube comprises a metal mesh.

5. The aural suction device according to claim 1, wherein the tubular handle is formed from a rigid material.

6. The aural suction device according to claim 5, wherein the rigid material is selected from the group consisting of plastic and steel.

7. The aural suction device according to claim 1, wherein the tubular handle comprises a rigid peripheral wall and the opening extends through a portion of the rigid peripheral wall.

8. The aural suction device according to claim 1, further comprising a vacuum source attached to the second open end.

9. The aural suction device according to claim 1, wherein the flexible tube comprises a flexible material.

10. The aural suction device according to claim 9, wherein the flexible material is selected from the group consisting of rubber and plastic.

11. The aural suction device according to claim 1 wherein the safety stop comprises a ring configured to adjustably grip an outer peripheral portion of the flexible tube.

12. The aural suction device according to claim 1 wherein the safety stop comprises a clamp configured to adjustably grip an outer peripheral portion of the flexible tube.

13. The aural suction device according to claim 12 wherein the clamp comprises a spring clamp.

14. The aural suction device according to claim 1, wherein the flexible tube has an inner diameter and the inner diameter is constant.

15. The aural suction device according to claim 1, wherein the tubular handle has an inner diameter and the inner diameter is constant.

16. The aural suction device according to claim 1, wherein the tubular handle has an inner diameter and the inner diameter is variable.

17. The aural suction device according to claim 7, wherein the opening is in fluid communication with the flexible tube, allowing for manual control of suction through the flexible tube.

18. An aural suction system, comprising:
an aural suction device including a tubular handle having first and second open ends and an opening between the first and second open ends, a flexible tube detachably connected to the first end of the tubular handle, and a safety stop adjustably secured to an outer surface of the flexible tube; and
a suction source attached to the second open end.

19. The aural suction device according to claim 18, wherein the suction source is a vacuum source.
